(19) United States
(12) Patent Application Publication
(10) Pub. No.: US 2018/0055108 A1
(43) Pub. Date: Mar. 1, 2018

(54) PAD DEVICE AND GLOVE FOR PROTECTION OF HYPOTHENAR REGION, AND ASSOCIATED SYSTEMS AND METHODS

(71) Applicant: EASTON BASEBALL / SOFTBALL INC., Thousand Oaks, CA (US)

(72) Inventors: Keenan Long, Sherman Oaks, CA (US); Ryan Polanco, Thousand Oaks, CA (US)

(21) Appl. No.: 15/253,515

(22) Filed: Aug. 31, 2016

(51) Int. Cl. A41D 13/08; A61F 5/30; A63B 71/14; A63B 59/00; A41D 31/00

(52) U.S. Cl. CPC A41D 13/082; A61F 5/30; A63B 2102/182; A63B 59/00; A41D 31/0011; A63B 71/143

(57) ABSTRACT

A pad device for protecting a hypothenar eminence region of a user's hand during a swing of a ball bat includes a digital portion having an opening configured to receive at least one of a user's fingers, and a hypothenar portion generally configured to coextend with the hypothenar eminence region of the user's hand. The pad device may include a viscoelastic material or a rate-dependent damping material. In other embodiments, a sports glove includes a glove base and a moldable hypothenar layer coupled to the glove base and positioned to generally coextend with a hypothenar eminence region of a user's hand. The moldable hypothenar layer may include an at least partially rigid moldable material configured to be molded into a form of at least one of the hypothenar eminence region of the user's hand or an end portion of a ball bat. The moldable material may include moldable thermoplastic.

### BACKGROUND

A baseball or softball batter typically swings a bat several times during a game or in practice or training. During a batter's swing, rapid acceleration and deceleration of the barrel and vibrations from impact with a ball result in strong forces that can damage the fibrous connective tissues, muscles, tendons, and ligaments of the batter's hands, and can cause blisters, callouses, bruises, open wounds, and even broken bones in the hand. A batter's maximum swing speed may be limited by the batter's ability to dissipate the forces experienced by the hand when decelerating a bat at the end of the swing.

Some batters wear a thin batting glove on one or both hands to try to reduce damage to their hands during a swing. But strong forces continue to be dissipated by the batter's hand, so damage can still result from rapid or repeated swings. Other batters may use a loose or floating cushioning item stuffed inside the glove with the batter's hand. But such a cushioning item may move unpredictably and cause distraction of the batter's attention away from an incoming pitch.

Some bats are available with asymmetric handles that seek to reduce the pressure on the hypothenar region of the hand using a handle shape that increases surface area. But many hitters do not like such bats because they disrupt the batter's proprioception and tactile gnosis. In other words, such bats may feel strange to a batter accustomed to a standard handle and circular bat knob, and consequently such bats may be yet another distraction from an incoming pitch.

Because of these disadvantages with existing technology, hand trauma associated with swinging a bat continues to be a problem for batters.

### SUMMARY

A pad device for protecting a hypothenar eminence region of a user's hand during a swing of a ball bat includes a digital portion having an opening configured to receive at least one of a user's fingers, and a hypothenar portion configured to generally coextend with the hypothenar eminence region of the user's hand. The pad device may include a viscoelastic material or a rate-dependent damping material. In other embodiments, a sports glove includes a glove base and a moldable hypothenar layer coupled to the glove base and positioned to generally coextend with a hypothenar eminence region of a user's hand. The moldable hypothenar layer may include an at least partially rigid moldable material configured to be molded into a form of at least one of the hypothenar eminence region of the user's hand and an end portion of a ball bat. The moldable material may include a moldable thermoplastic material. Embodiments of the present technology protect a user's hypothenar region or hypothenar eminence region during a swing of a ball bat, while minimizing reductions in tactile gnosis, thereby enabling a user to swing a ball bat faster and harder.

Other features and advantages will appear hereinafter. The features described above may be used separately or together, or in various combinations of one or more of them.

### BRIEF DESCRIPTION OF THE DRAWINGS

In the drawings, wherein the same reference number indicates the same element throughout the views:

FIG. 1 illustrates a pad device for protecting a hypothenar region or hypothenar eminence region of a user's hand in accordance with an embodiment of the present technology.

FIG. 2 illustrates the pad device of FIG. 1 as worn on a user's hand.

FIG. 3 illustrates the pad device of FIGS. 1 and 2 in use when a user grips the handle of a ball bat.

FIG. 4 illustrates a pad device in accordance with another embodiment of the present technology.

FIG. 5 illustrates a pad device in accordance with another embodiment of the present technology.

FIG. 6 illustrates a palmar view of a glove assembly in accordance with another embodiment of the present technology.

FIG. 7 illustrates a partial cross-sectional view of a hypothenar region of the glove assembly illustrated in FIG. 6 in accordance with an embodiment of the present technology.

FIG. 8 illustrates the glove assembly generally illustrated in FIG. 6 in use or as worn by a user in accordance with an embodiment of the present technology.

FIG. 9 illustrates a glove assembly in accordance with an embodiment of the present technology in which a moldable layer interposed between an outer hypothenar layer and a glove base has been molded into a form of a bat handle and knob and to the user's hand beneath the glove base.

FIG. 10 illustrates another embodiment of the present technology in which a moldable element may be held directly in a user's hand rather than contained within a glove assembly.

FIG. 11 illustrates the moldable element of FIG. 10 in a user's hand after the element has been molded into form.

### DETAILED DESCRIPTION

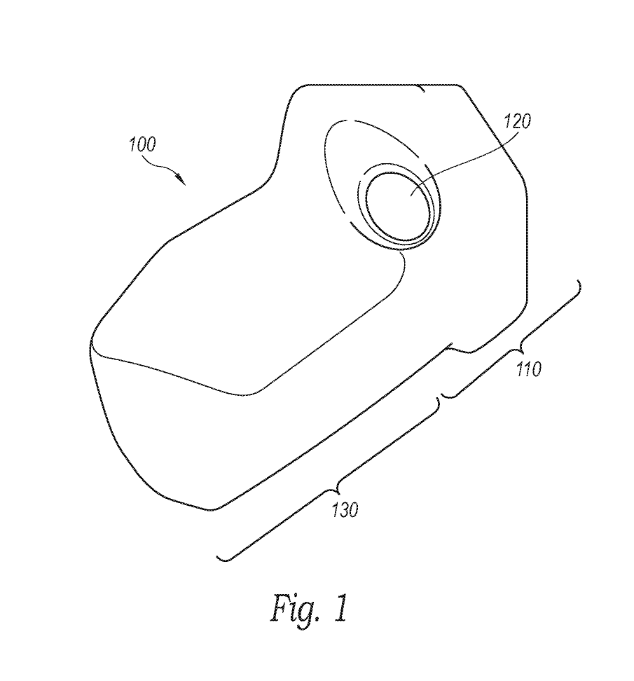

The present technology is directed to pad devices and gloves for protection of a hypothenar eminence region of a user's hand, and associated systems and methods. Various embodiments of the technology will now be described. The following description provides specific details for a thorough understanding and an enabling description of these embodiments. One skilled in the art will understand, however, that the invention may be practiced without many of these details. Additionally, some well-known structures or functions may not be shown or described in detail so as to avoid unnecessarily obscuring the relevant description of the various embodiments. Accordingly, the technology may include other embodiments with additional elements or without several of the elements described below with reference to FIGS. 1-11, which illustrate examples of the technology.

The terminology used in the description presented below is intended to be interpreted in its broadest reasonable manner, even though it is being used in conjunction with a detailed description of certain specific embodiments of the technology. Certain terms may even be emphasized below; however, any terminology intended to be interpreted in any restricted manner will be overtly and specifically defined as such in this detailed description section.

Where the context permits, singular or plural terms may also include the plural or singular term, respectively. Moreover, unless the word “or” is expressly limited to mean only a single item exclusive from the other items in a list of two or more items, then the use of “or” in such a list is to be interpreted as including (a) any single item in the list, (b) all of the items in the list, or (c) any combination of items in the list. Further, unless otherwise specified, terms such as “attached” or “connected” are intended to include integral connections, as well as connections between physically separate components.

Specific details of several embodiments of the present technology are described herein with reference to baseball or softball. The technology may also be used in other sports or industries when the disclosed features or advantages are desirable, such as other sports involving implements including lacrosse sticks, hockey sticks, cricket bats, field hockey sticks, golf clubs, or other implements. Conventional aspects of gloves may be described in reduced detail herein for efficiency and to avoid obscuring the present disclosure of the technology.

### Finger-Secured Hypothenar Pad

FIG. 1 illustrates a pad device or pad 100 for protecting a hypothenar region or hypothenar eminence region of a user's hand in accordance with an embodiment of the present technology. The pad 100 may have a digital portion 110 positioned to receive one or more of a user's fingers in a finger opening 120 in order to couple the pad 100 to the user's hand. For example, a user's little finger or ring finger may be positioned in the opening 120. The pad 100 may further include a hypothenar portion 130 sized and shaped to be generally coextensive with a hypothenar region or hypothenar eminence region of the user's hand when worn by the user. Although a generally oblong shape is illustrated in FIG. 1, any suitable shape or size may be used to coextend with the user's hypothenar region or hypothenar eminence region without unduly extending to other areas of a user's hand.

In some embodiments, the digital portion 110 may extend along a length of the pad 100 in a direction opposite the hypothenar portion 130, such that the digital portion 110 extends beyond the finger opening 120. In other embodiments, the digital portion 110 may be suitably sized or shaped to encircle one or more of the user's fingers with only minimal material extending from the digital portion 110 opposite the hypothenar portion 130. For example, the digital portion 110 may be formed from ring elements or it may include ring elements attached to the hypothenar portion 130.

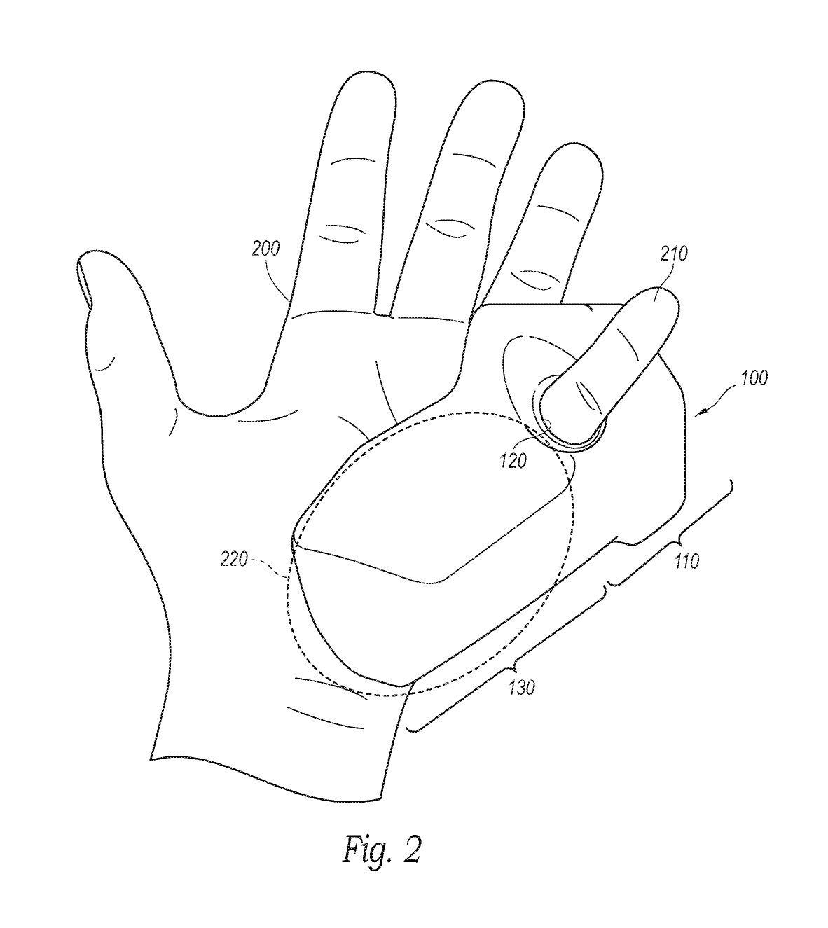

FIG. 2 illustrates the pad 100 as worn on a user's hand 200 in accordance with an embodiment of the present technology. In use, a user may secure the pad 100 to the hand 200 by positioning a little finger 210 in the opening 120. The hypothenar portion 130 is sized and configured to generally coextend with the hypothenar region or hypothenar eminence region of the user's hand (generally located within area 220 in FIG. 2). In some embodiments, the pad 100 does not extend to cover other parts of the user's hand in order to maintain sufficient tactile gnosis with such other parts of the hand 200 when gripping a ball bat or other implement. For example, the pad 100 may not cover portions of a palm region of the user's hand beyond the hypothenar eminence region.

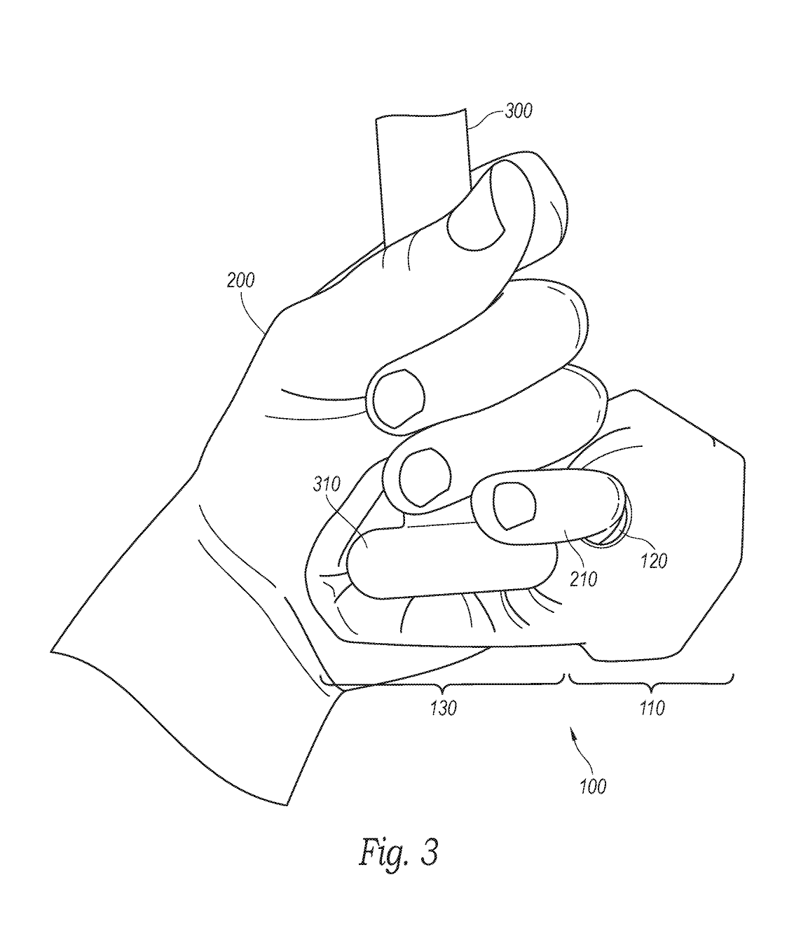

FIG. 3 illustrates the pad 100 in use when a user grips the handle 300 of a ball bat using the pad 100. In order to increase leverage in a swing, a user may choose to grip the bat low on the handle 300 towards the knob 310, potentially to include the knob 310 itself. In such a grip, the hypothenar portion 130 of the pad 100 protects the user's hypothenar eminence region as generally represented by area 220 in FIG. 2.

The pad 100 may be formed using a rate-dependent damping material such as a suitable viscoelastic material. For example, the rate-dependent damping material may include thermoplastic urethane (TPU) or urethane foam material such as PORON® XRD® or thermo-pressed polyurethane composite material available from Taiwan Kurim Enterprise Co., or other suitable thermoplastic urethane or urethane foam materials. In some embodiments, the rate-dependent damping material may include clay. The rate-dependent damping material forming the pad 100 may have a thickness within the range of approximately 0.05 inches and 0.5 inches. For example, it may have a thickness of approximately 0.25 inches. Such a rate-dependent damping material tends to conform to the shape of a user's hand 200 and to the shape of the bat handle 300 and the knob 310 when the user gradually grips the handle 300 and the knob 310, but is relatively slow to deform when receiving an impact or impulse force. Accordingly, the pad 100 may generally maintain its shape and thickness when subjected to large impulse forces such as reaction forces from impact with a ball during a swing or reaction forces from decelerating the bat at the end of a swing. The pad 100, therefore, protects the user's hand by absorbing such impulse forces.

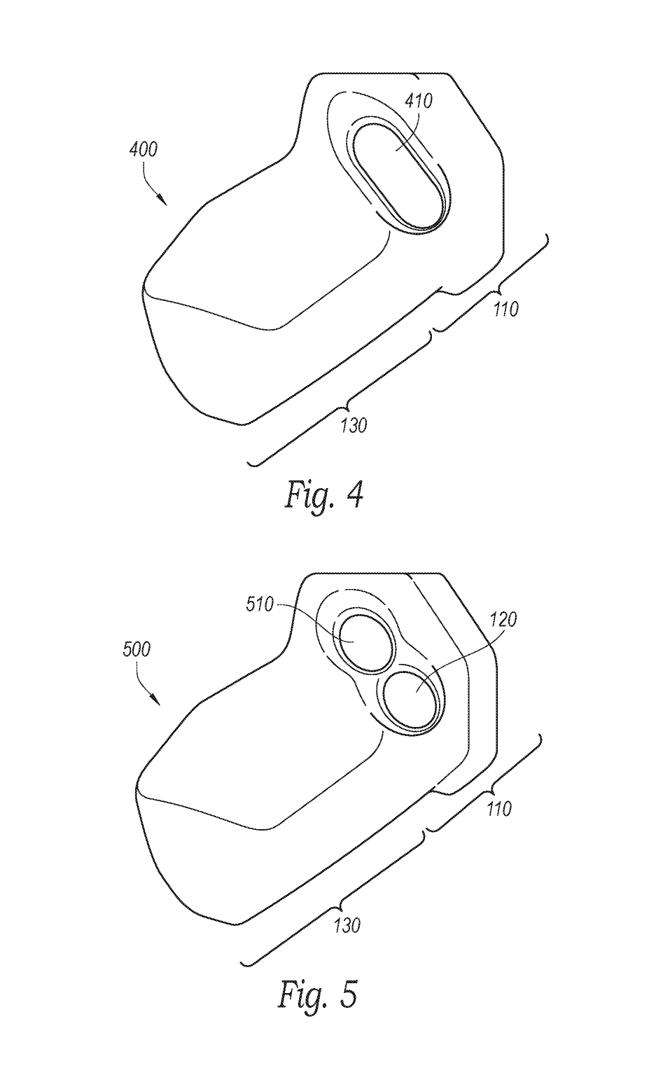

FIG. 4 illustrates a pad 400 in accordance with another embodiment of the present technology. The pad 400 may be generally similar to the pad 100 described above with regard to FIGS. 1-3, except that the pad 400 has a finger opening 410 shaped and sized to receive a user's ring and little fingers together.

FIG. 5 illustrates a pad 500 in accordance with another embodiment of the present technology. The pad 500 may be generally similar to the pad 100 described above with regard to FIGS. 1-3, except that in addition to an opening 120 for a little finger, the pad 500 further includes a separate opening 510 for a user's ring finger. In the embodiments shown in FIGS. 4 and 5, the pads (400, 500) may be used in a similar manner as the pad 100 illustrated in FIGS. 1-3 to provide protection for the hypothenar region or hypothenar eminence region of a user's hand.

In some embodiments, the digital portions 110 of the pads 100, 400, 500 may be shaped and sized such that the pads 100, 400, 500 fit under a traditional batting glove. In other embodiments, the pads 100, 400, 500 may be shaped and sized to be used alone or without a traditional batting glove.

In embodiments of the present technology, a pad 100, 400, or 500 may be shaped and sized to protect the hypothenar region or hypothenar eminence region of the palm of the hand 200 (for example, the region identified as area 220 in FIG. 2) adjacent to the proximal end (for example, the knob 310) of the bat while being coupled to a ring finger, a little finger, or both a ring and little finger of the batter's hand. Because the pads 100, 400, 500 may be shaped and sized to protect generally only the hypothenar eminence region of the user's hand, the pads protect the area of the hand that is most likely to suffer injury while minimizing disruption of the batter's tactile gnosis during a swing (for example, by not protecting portions of the user's palm beyond the hypothenar region or hypothenar eminence region).

Glove with Moldable Hypothenar Intermediate Layer

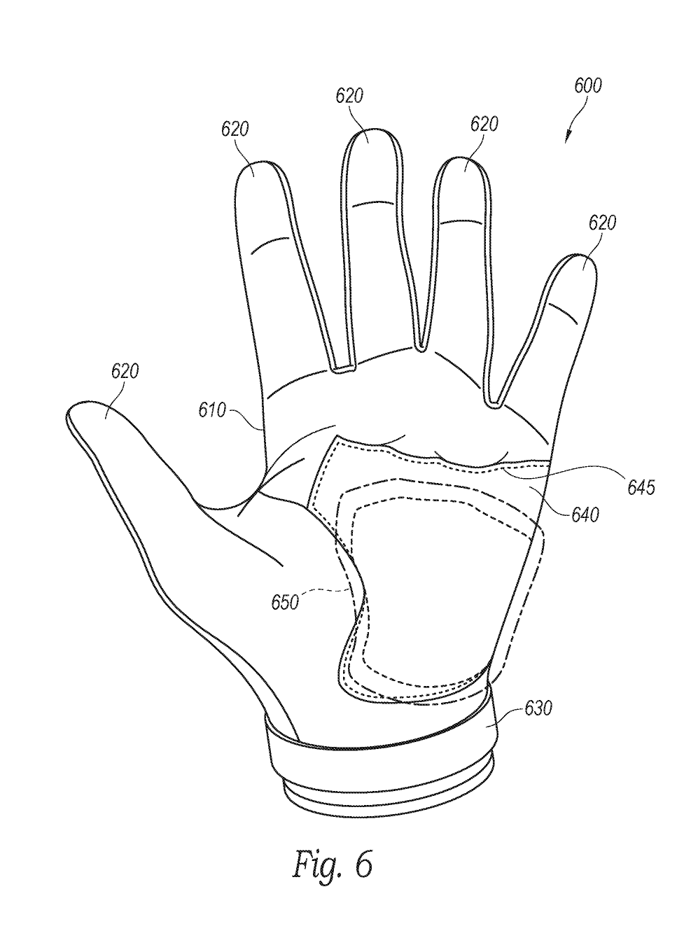

FIG. 6 illustrates a palmar view of a glove assembly 600 in accordance with another embodiment of the present technology. The glove assembly 600 includes a glove base 610 which may be formed as a glove shaped to accommodate a user's left or right hand. The glove base 610 may include thumb and finger portions 620 for accommodating a user's thumb and fingers. Although the glove base 610 may resemble a full glove in some embodiments, in other embodiments the glove base 610 may have only partial or even minimal finger portions 620. For example, the glove base 610 may be generally fingerless. The glove base 610 may be formed from natural leather, synthetic leather, sheepskin, goatskin, microfiber, cloth, fabric, or other suitable materials.

The glove assembly 600 may also include a fastening strap 630. The fastening strap 630 may be a conventional fastening strap for retaining the glove assembly 600 on a user's hand. For example, the fastening strap 630 may be formed from elastic, VELCRO® hook-and-loop straps, or an arrangement of straps and buckles.

An outer hypothenar layer 640 may be stitched, glued, riveted, or otherwise suitably fastened to the glove base 610 in the glove assembly 600, or it may be an integral region of the glove base. For example, the outer hypothenar layer 640 may be fastened to the glove base 610 around a perimeter 645 of the outer hypothenar layer 640. The outer hypothenar layer 640 may be sized, shaped, or otherwise configured to generally coextend with or generally overlay a hypothenar region or hypothenar eminence region of the user's hand when the user wears the glove assembly 600. For example and for illustration, the hypothenar region or hypothenar eminence region of the user's hand may generally be located within the area 650 illustrated in FIG. 6. In some embodiments, the outer hypothenar layer 640 does not coextend with substantially all of the palmar region of the glove base 610 and may be limited generally to the hypothenar region or hypothenar eminence region of the user's hand. As will be explained below, such a limited region allows for enhanced protection of the user's hypothenar region or hypothenar eminence region without reducing tactile gnosis in other regions of the hand.

In embodiments in which the outer hypothenar layer 640 is fastened to the glove base 610 around a perimeter 645 of the outer hypothenar layer 640, the outer hypothenar layer 640 and the glove base 610 form a pocket area, opening, or space between the outer hypothenar layer 640 and the glove base 610.

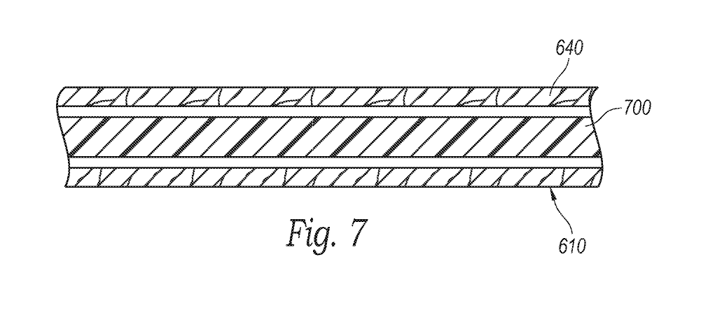

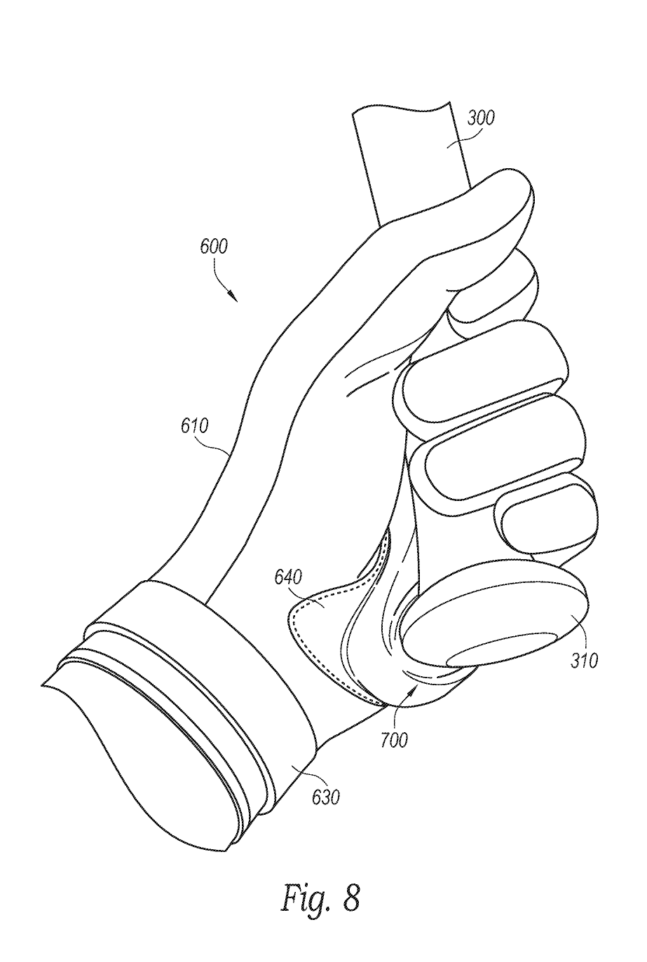

FIG. 7 illustrates a partial cross-sectional view of a hypothenar region of the glove assembly 600. A moldable hypothenar intermediate layer 700 resides in a space between the glove base 610 and the outer hypothenar layer 640. The moldable layer 700 may generally coextend with or generally overlay the hypothenar region or hypothenar eminence of a user's hand when the user is wearing the glove assembly 600.

The moldable layer 700 may be formed from a moldable material that “learns” a shape and retains that shape for an extended time or indefinitely. For example, the moldable layer 700 may be formed from a thermoplastic or thermoformable material that can be heated to become flexible to be formed into a shape. Upon cooling, the material remains molded in such a shape. The moldable layer 700 may have a thickness within the range of approximately one-tenth of an inch to one-half of an inch, or it may have any other suitable thickness.

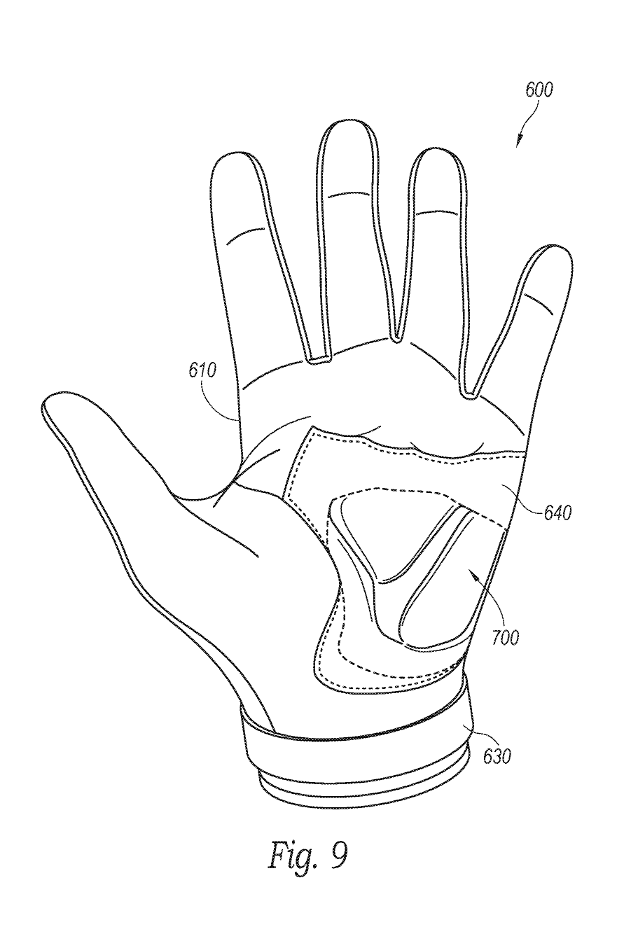

FIG. 8 illustrates the glove assembly 600 in use or as worn by a user. Initially, to customize the fit and form of the glove assembly 600 including the moldable layer 700, a user of the glove assembly 600 may heat the glove assembly 600 or the portion of the glove assembly 600 having the moldable layer 700 to put the moldable layer 700 in condition for molding. The user may provide heat with heated water, hot air (such as from a heat gun or hair dryer), or other forms of radiative or convective heat. When the moldable layer 700 is in a softened moldable form, the user may don the glove assembly 600 and grasp the bat handle 300 (and optionally the knob 310) as desired for a given hitting style to allow the moldable layer 700 to form into a shape corresponding to the user's hand on one side and to the bat handle 300 and knob 310 on the other side. As the moldable layer 700 cools, it retains its formed shape.

In some embodiments, a user may swing the ball bat several times while the moldable layer 700 remains warm or heated in order to simulate play to form the layer 700 into a favorable custom form that is retained when the layer 700 is cooled or cured. For example, FIG. 9 illustrates a glove assembly 600 in accordance with an embodiment of the present technology in which the moldable layer 700 (in the space interposed between the outer hypothenar layer 640 and the glove base 610) has been molded into the form of the bat handle 300 and knob 310 (illustrated in FIG. 8) and to the user's hand beneath the glove base 610. Once in this molded configuration, the user may use the glove assembly 600 during play.

The inventors discovered that such a glove assembly including a molded thermoplastic (or other material) layer generally conforming and coextending with a user's hypothenar region or hypothenar eminence region would protect the user's hand by dissipating force in an improved manner relative to a standard batting glove. Once molded into a hardened custom form, the layer 700 mates with the user's hand and with the bat handle 300 and knob 310, increasing the contact area and reducing pressure on vulnerable areas of the hand such as the hypothenar region. Accordingly, the technology disclosed herein reduces the risk of hand trauma during a swing. The technology may also enable the user to swing faster or harder than when using conventional batting gloves. For example, a user may be able to swing harder because the technology allows increased ability to decelerate the bat by dissipating the damaging forces associated with the deceleration portion of the swing.

The present technology allows for improved swing speed and protection from hand trauma while avoiding excessive loss of tactile gnosis. For example, the layer 700 may be shaped and sized to be positioned only in the hypothenar region or hypothenar eminence region of the user's palm, leaving the remainder of the hand capable of tactile perception of the bat. And the layer 700 is at least somewhat rigid when formed and cooled, so it will not deform through a swing, thereby limiting potential reductions in tactile gnosis. Yet another advantage of the present technology is that the layer 700 is discreet, such that the glove assembly 600 may be styled to aesthetically resemble traditional gloves.

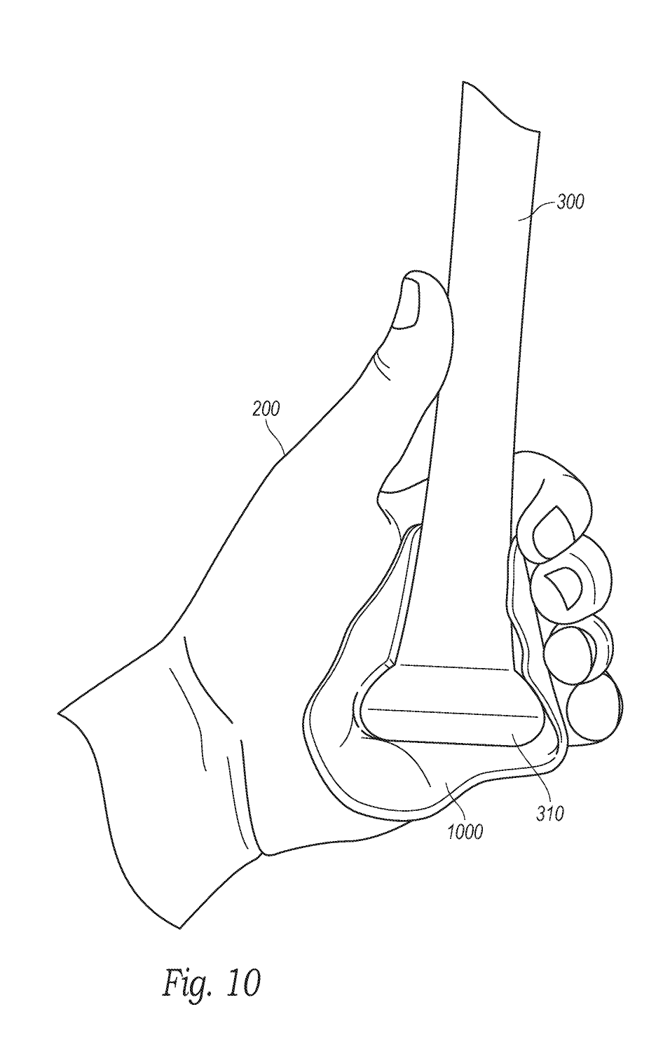

FIG. 10 illustrates another embodiment of the present technology. A moldable element 1000 may be similar to the moldable layer 700 described above with regard to FIGS. 7-9, but it may be held directly in a user's hand rather than contained within a glove assembly. In such an embodiment, the moldable element 1000 may be molded by a user after being softened by heat as described above, and the user may grasp or swing a bat several times until the moldable element 100 cools into form.

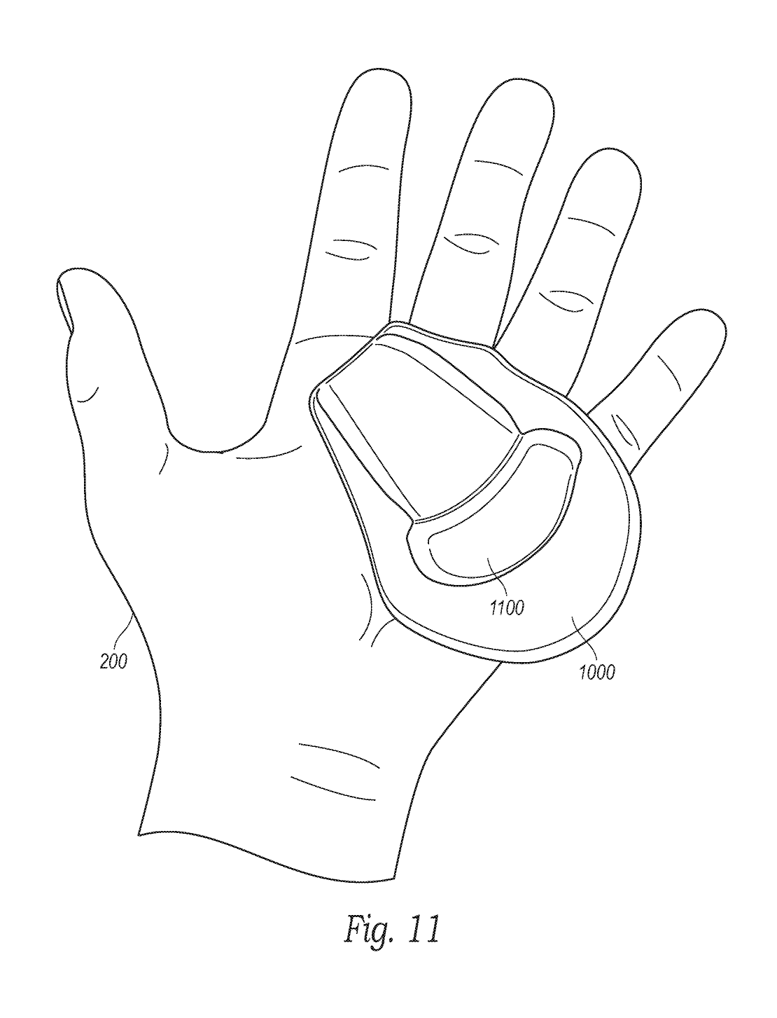

FIG. 11 illustrates the moldable element 1000 in a user's hand after it has been molded into form. An indentation 1100 remains in the moldable element 1000 after the molding process is complete. The moldable element 1000 may be configured such that it does not remain coupled to the ball bat on its own.

The technology disclosed herein provides a pad device for protecting a hypothenar region or hypothenar eminence region of a user's hand and a glove with a moldable hypothenar intermediate layer for protecting the hypothenar region or hypothenar eminence region. This technology allows batters to make more swings at maximum effort while reducing the risk of damage to the hand's hypothenar region. The technology disclosed herein may be used in other industries or other sports involving implements, such as lacrosse, hockey, cricket, field hockey, or golf. In various embodiments, a variety of straps, buckles, or other devices may be used to retain the technology on a user's hand. In addition, in various embodiments, a variety of suitable materials may be used.

From the foregoing, it will be appreciated that specific embodiments of the disclosed technology have been described for purposes of illustration, but that various modifications may be made without deviating from the technology, and elements of certain embodiments may be interchanged with those of other embodiments. For example, in some embodiments, the moldable layer 700 may be positioned in a pocket or opening of a glove base rather than interposed between layers of a glove assembly. In other embodiments, the moldable layer 700 may be an outermost layer of a glove assembly, or may be coupled to a glove assembly, with or without a covering layer on the moldable layer 700.

Further, while advantages associated with certain embodiments of the disclosed technology have been described in the context of those embodiments, other embodiments may also exhibit such advantages, and not all embodiments need necessarily exhibit such advantages to fall within the scope of the technology. Accordingly, the disclosure and associated technology may encompass other embodiments not expressly shown or described herein, and the invention is not limited except as by the appended claims.

What is claimed is:

1. A pad device for protecting a hypothenar eminence region of a user's hand during a swing of a ball bat, comprising:
a digital portion having a finger opening configured to receive at least one of a user's fingers; and
a hypothenar portion extending from the digital portion and configured to generally coextend with the hypothenar eminence region of the user's hand.

2. The pad device of claim 1 wherein the hypothenar portion comprises a viscoelastic material.

3. The pad device of claim 2 wherein the viscoelastic material includes a thermoplastic urethane (TPU) material.

4. The pad device of claim 2 wherein the viscoelastic material includes a clay material.

5. The pad device of claim 1 wherein the finger opening is configured to receive only a little finger of a user.

6. The pad device of claim 1 wherein the finger opening is configured to receive only a ring finger of a user.

7. The pad device of claim 1 wherein the finger opening is a first finger opening and the pad device further comprises a second finger opening configured to receive another of the user's fingers.

8. The pad device of claim 1 wherein the finger opening is configured to receive only a ring finger and a little finger of a user.

9. The pad device of claim 1 wherein the hypothenar portion coextends only with the hypothenar eminence region of a user's hand.

10. A sports glove comprising:
a glove base configured to generally contain at least a portion of a user's hand; and
a moldable hypothenar layer coupled to the glove base and positioned to generally coextend with a hypothenar eminence region of a user's hand, wherein
the moldable hypothenar layer includes an at least a partially rigid moldable material configured to be molded into a form of at least one of the hypothenar eminence region of the user's hand or an end portion of a ball bat.

11. The sports glove of claim 10 wherein the moldable material includes moldable thermoplastic.

12. The sports glove of claim 10 wherein the glove base includes an outer hypothenar layer attached to the glove base and configured to generally coextend with the hypothenar eminence region of a user's hand.

13. The sports glove of claim 12 wherein the outer hypothenar layer is integrally formed with the glove base.

14. The sports glove of claim 12 wherein the moldable hypothenar layer is positioned between the outer hypothenar layer and the glove base.

15. The sports glove of claim 10 wherein the moldable hypothenar layer does not generally coextend with an entirety of a palm region of a user's hand.

16. The sports glove of claim 10 wherein the moldable hypothenar layer has a thickness within a range of approximately one-tenth of an inch to one-half of an inch.

17. A grip element for a ball bat, the grip element comprising:
a moldable element configured to protect at least a portion of a user's hand during a swing of a ball bat, the moldable element including an at least partially rigid, moldable material configured to be molded into a form of both the hypothenar eminence region of the user's hand and an end portion of the ball bat.

18. The grip element of claim 17 wherein the end portion of the ball bat comprises a handle and a knob.

19. The grip element of claim 17 wherein the moldable element is configured to be positioned inside of a sports glove.

20. The grip element of claim 17 wherein the grip element is not configured to be permanently or semi-permanently mounted to a handle of the ball bat.
